(19) United States
(12) Patent Application Publication
(10) Pub. No.: US 2004/0225326 A1
(43) Pub. Date: Nov. 11, 2004

(54) Apparatus for the detection of restenosis

(76) Inventors: Mike L. Weiner, Webster, NY (US); Patrick R. Connelly, Rochester, NY (US)

Correspondence Address: HOWARD J. GREENWALD P.C., 349 W. COMMERCIAL STREET SUITE 2490, EAST ROCHESTER, NY, 14445-2408, US

(21) Appl. No.: 10/836,686

(22) Filed: Apr. 30, 2004

Related U.S. Application Data
(63) Continuation-in-part of application No. 10/208,288, filed on Jul. 30, 2002.
Application No. 10/208,288 is a continuation of application No. 10/131,361, filed on Apr. 24, 2002.
Application No. 10/131,361 is a continuation of application No. 09/918,078, filed on Jul. 30, 2001, now Pat. No. 6,743,190.
Application No. 10/131,361 is a continuation of application No. 09/918,076, filed on Jul. 30, 2001, now Pat. No. 6,793,642.
Application No. 09/918,076 is a continuation of application No. 09/850,250, filed on May 7, 2001, now Pat. No. 6,488,704.

(51) Int. Cl. A61N 1/00

(52) U.S. Cl. 607/2

(57) ABSTRACT

A medical apparatus is disclosed for determining the degree of restenosis of a stent comprising a stent, an energy transmitter, an energy receiver, and a processor to compare the transmitted energy and the received energy.

### CROSS-REFERENCE TO RELATED PATENT APPLICATIONS

This application is a continuation-in-part of co-pending patent application U.S. Ser. No. 10/208,288, filed on Jul. 30, 2002, which in turn is a continuation of co-pending patent application U.S. Ser. No. 10/131,361 filed on Apr. 240, 2002, which in turn is a continuation of co-pending patent applications U.S. Ser. No. 09/918,078 and 09/918,076, both filed on Jul. 30, 2001, which in turn were continuations of patent application U.S. Ser. No. 08/850,250 filed on May 7, 2001, which issued as U.S. Pat. No. 6,488,704 on Dec. 3, 2002.

### FIELD OF THE INVENTION

This invention relates, in one embodiment, to methods for the detection of stenosis and restenosis, and more particularly to a stent adapted to detect restenosis.

### BACKGROUND OF THE INVENTION

Medical stents are commonly used to treat blocked or obstructed lumens, such as blood vessels. Such an obstruction is often referred to as stenosis. Stents find uses in a number of medical fields, including cardiovascular, gastroenterology, urology, and the like.

One serious deficiency of stent technology is the reocclusion of the lumen by restenosis. After a stent has been inserted, there is a tendency for smooth muscle cells and/or plaque to proliferate on the surface of the stent, thus causing a blockage of the lumen.

Current treatments for restenosis generally involve invasive procedures wherein plaque buildup is physically removed. An alternative procedure involves the complete replacement of the blocked stent with a replacement stent.

U.S. Pat. No. 6,015,387 to Schwartz et al. describes and claims a stent adapted to measure blood flow. “The device includes a piezoelectric crystal for generating an ultrasonic wave that is directed toward the blood vessel. The same or a second piezoelectric crystal is employed to detect the reflected vibrational wave from the blood vessel and produce an RF signal that is indicative of blood flow within the blood vessel.” The patent also teaches that the stent “can also provide a therapeutic function by applying heat or vibration to the blood to inhibit restenosis. In one embodiment, a feed-back control loop regulates the therapeutic functions based on measurements of blood flow.” Thus, this patent teaches one method for indirectly measuring restenosis, but fails to teach or suggest a method for the direct measurement of plaque accumulation. The contents of U.S. Pat. No. 6,015,387 are hereby incorporated by reference.

U.S. Pat. No. 6,170,488 to Spillman et al. discloses a method for detecting the status of an implanted medical device based on acoustic harmonics. “For example, the presence of harmonics in a stent 32 may increase or decrease as a function of the degree of restenosis which occurs within the stent. Thus, by monitoring the presence of harmonics over the course of periodic testing (e.g., trending), it is possible to track the build-up of restenosis.” Thus, this patent teaches one method for indirectly measuring plaque accumulation, but fails to teach or suggest a method for the direct measurement of restenosis. Additionally, a significant amount of restenosis must occur before the acoustic harmonics of the stent are significantly altered. Frequently exposing the stent to vibration energy also causes damage to the stent and the surrounding issues. The contents of U.S. Pat. No. 6,170,488 are hereby incorporated by reference.

U.S. Pat. No. 6,200,307 to Kasinkas et al. teaches a method for the treatment of in-stent restenosis. The specification teaches “. . . method of treating in-stent restenosis by applying radiation to the smooth muscle cells which have grown within or around a stent implant in a manner that does not substantially damage the surrounding lumen wall or the stent itself, while resulting in a reduction of smooth muscle cell mass.” The radiation is introduced into a stent by way of a flexible catheter. Thus, this patent teaches one method for removing plaque accumulation, but fails to teach or suggest a means for detecting the degree of plaque accumulation. The prior art also fails to teach or suggest the use of a stent that removes restenosis without the aid of an external device. The contents of U.S. Pat. No. 6,200,307 are hereby incorporated by reference.

U.S. Pat. No. 6,488,704 to Connelly teaches et al. describes a stent adapted to function as a flow cytometer. The implantable stent contains “ . . . several optical emitters located on the inner surface of the tube, and several optical photodetectors located on the inner surface of the tube.” As labeled particles pass through the stent, the optical emitters and photodetectors are capable of detecting the labeled cells. Thus, this patent teaches one method for detecting particles flowing through a stent. The contents of U.S. Pat. No. 6,488,704 are hereby incorporated by reference.

U.S. Pat. Nos. 6,491,666 and 6,656,162 to Santini et al each disclose and claim a medical stent adapted to release molecules in response to a signal from a microchip which is attached to the surface of the stent. The integration of microchip devices into stents is described in this patent. In one embodiment, the molecules that are released by the stent are anti-restenosis drugs. The contents of U.S. Pat. Nos. 6,491,666 and 6,656,162 are hereby incorporated by reference.

It is an object of this invention to provide at least one of the following: a stent capable of directly detecting the presence of plaque within the stent, a stent capable of removing plaque within the stent, and a process for the direct detection of plaque within a stent.

### SUMMARY OF THE INVENTION

In accordance with the present invention, there is provided an apparatus and method for the detection of in-stent restenosis by comparison of the intensity of a transmitted wave and a received wave. When a fluid is flowing through an unblocked stent, a baseline measurement is made. As the stent accumulates plaque, the intensity of the received wave slowly decreases relative to the intensity of the transmitted wave. This decrease can be optionally coupled to a therapeutic treatment to inhibit the restenosis.

The technique described above is advantageous because it more simple than the prior art stents. The use of low intensity electromagnetic waves does not cause damage to the stent or the surrounding issue. Thus, the technique can be used frequently or even continuously to monitor the degree of restenosis. Additionally, the invention allows the monitoring of restenosis without using invasive techniques.

### BRIEF DESCRIPTION OF THE DRAWINGS

The invention will be described by reference to the following drawings, in which like numerals refer to like elements, and in which:

FIG. 1A is a cut away view of an apparatus that uses one embodiment of the instant invention;

FIG. 1B is an end view of a stent;

FIG. 1C is an end view of a stent suffering from restenosis;

FIG. 2 is a cross sectional view of one embodiment of the invention;

FIG. 3 is a cross sectional view of one embodiment of the invention showing the transmission of parallel energy in one direction;

FIG. 4 is an end view of a stent similar to that shown in FIG. 3;

FIG. 5 is a cross sectional view of one embodiment of the invention showing the transmission of energy through plaque;

FIG. 6 is an end view of a stent similar to that shown in FIG. 5;

FIG. 7 is a cross sectional view of one embodiment of the invention showing the transmission of parallel energy in multiple directions;

FIG. 8 is an end view of a stent similar to that shown in FIG. 7;

FIG. 9 is a cross sectional view of one embodiment of the invention showing the transmission of non-parallel energy;

FIG. 10 is an end view of a stent similar to that shown in FIG. 9;

FIG. 11 is an end view of a stent similar showing communication with a remote unit;

FIG. 12 is a flow diagram illustrating one process of the invention; and

FIG. 13 is a flow diagram illustrating another process of the invention.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by the appended claims.

### DESCRIPTION OF THE PREFERRED EMBODIMENTS

In describing the present invention, a variety of terms are used in the description.

The term “stent” refers to a cylinder or scaffold made of metal or polymers that may be permanently implanted into a blood vessel following angioplasty procedure. Reference may be had to U.S. Pat. No. 6,190,393, the ensure disclosure of which is hereby incorporated by reference. The term stent also refers to such a cylinder or scaffold used in lumens other than blood vessels.

The term “stenosis” refers to the constriction or narrowing of a passage, duct, stricture, or lumen, such as a blood vessel. “Restenosis” refers to the reoccurrence of stenosis in a lumen (or implanted medical device).

The term “baseline value” refers to the measurement taken at specified period in time, which is later to be used as a reference point for comparison to a second measurement. Baseline measurements are typically taken when the stent is in pristine condition. The baseline measurement allows the user to correct for energy reading variations due to the fluids that may fill the stent. A certain amount of deviation from the baseline reading is acceptable, as this may account for the inhomogeneity of many fluids.

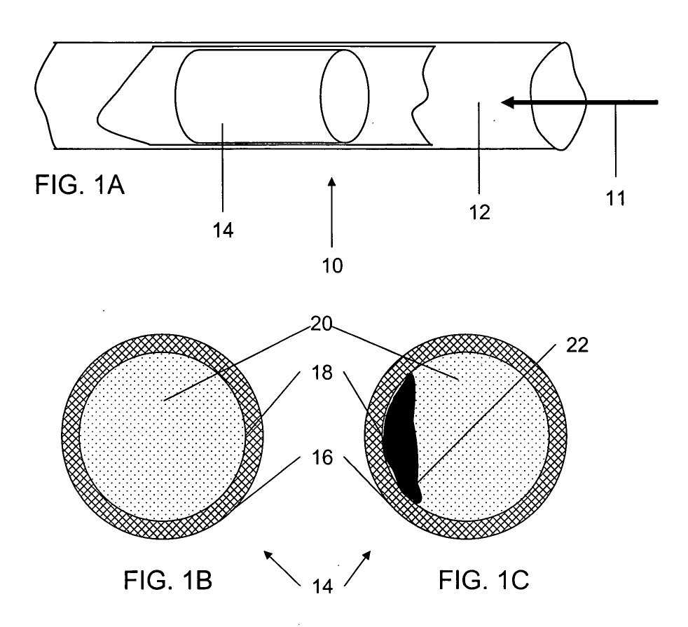

FIG. 1A is a cut away view of an apparatus that utilizes one embodiment of the instant invention. In the embodiment depicted in FIG. 1A, apparatus 10 comprises stent 14 is disposed within lumen 12. In one embodiment, a fluid flows through lumen 12 in the direction of arrow 11. In one embodiment, stent 14 is substantially flexible. In another embodiment, stent 14 is substantially inflexible.

FIG. 1B is a cross sectional view of stent 14. In the embodiment depicted in FIG. 1B, stent 14 comprises a cavity 20, an outer wall 16, and an inner wall 18. In the embodiment depicted in FIG. 1C, stent 14 suffers from the buildup of plaque 22. This restenosis causes the obstruction of cavity 20. In one embodiment, inner wall 18 is optional. When the stent is implanted within a living organism, it is preferable that the tissue-contacting surfaces be biocompatible. In the embodiment depicted in FIG. 1B, outer wall 16 and inner wall 18 are biocompatible. In one embodiment, the wall is comprised of one or more of the biocompatible materials disclosed in U.S. Pat. No. 6,124,523, the contents of which are hereby incorporated by reference. In another embodiment, the wall is comprised of polytetrafluoroethylene. In additional embodiments, other fluorinated plastics are used.

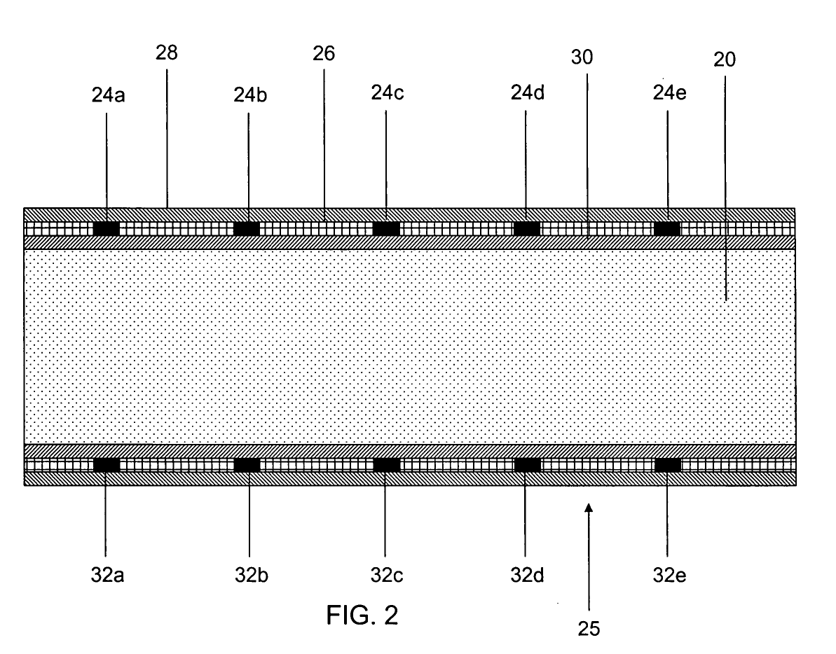

FIG. 2 is a cross sectional view of another embodiment of the invention. In the embodiment depicted in FIG. 2, stent 25 comprises cavity 20, outer wall 28, inner wall 30, and middle layer 26. Disposed within middle layer 26 are elements 24a to 24e and 32a to 32e. In one embodiment, elements 24a to 24e function as transmitters of electromagnetic energy while elements 32a to 32e function as receivers of electromagnetic energy. In another embodiment, elements 24a to 24e and 32a to 32e function as both transmitters and receivers of electromagnetic energy. In one embodiment, the transmitters 24 and receivers 32 are comprised of one or more of the transmitters and receivers disclosed in U.S. Pat. No. 6,488,704. In another embodiment, transmitters 24 and receivers 32 are comprised of VCSEL (vertical cavity surface emitting lasers). Reference may had, for example, or U.S. Pat. No. 6,686,216 (“Electro-optical transceiver system with controlled lateral leakage and method of making it”). In one embodiment of the invention, elements 24a to 24e function as transmitters of vibrational energy. In another embodiment, both vibrational and electromagnetic energy is generated. In another embodiment, an energy wave is generated using a piezoelectric crystal. In this embodiment, the energy wave is a vibrational energy wave. In yet another embodiment, element 24a emits a first type of energy while element 24b emits a second type of energy. By way of illustration, and not limitation, element 24a may emit light of a given wavelength, while element 24b emits light of a second wavelength. Alternatively or additionally, one such transmitting element may emit electromagnetic energy, while a second element emits vibrational energy. In one embodiment, the transmitting elements are activated simultaneously. In another embodiment, the elements are activated sequentially.

Receivers 32 may be comprised of a variety of materials. In one embodiment, the receiver element is a traditional antenna that is commonly utilized by one skilled in the art. In one embodiment, the receiver is a coil or circuit imposed on or within walls 26, 28, and/or 30. Reference may be had to U.S. Pat. Nos. 5,737,699 and 5,627,552 (“Antenna structure for use in a timepiece”), U.S. Pat. No. 5,495,260 (“Printed circuit dipole antenna”), U.S. Pat. No. 5,206,657 (“Printed Circuit Radio Frequency Antenna”), U.S. Pat. No. 6,650,301 (“Electrically conductive patterns, antennas, and methods of manufacture”), U.S. Pat. No. 5,535,304 (“Optical transceiver unit”), U.S. Pat. No. 4,549,314 (“Optical communication apparatus”), and the like. In another embodiment, the receiving elements are those described in U.S. Pat. No. 5,602,647 (“Apparatus and method for optically measuring concentrations of components”). The content of each of these patents is hereby incorporated by reference.

In the embodiment shown in FIG. 2, only ten such elements are shown. The embodiment has been illustrated as such only to simplify the illustration and prevent overcrowding of the drawing. As would be apparent to one skilled in the art, any number of transmitting and receiving elements may be used. In one embodiment, there is at least 1 such element per square centimeter surface area of inner wall 30. In another embodiment, there is at least 1 such element per square millimeter surface area. It is advantageous to place enough transmitting and receiving elements within stent 38 to ensure that any restenosis that begins to occur is detected. In one embodiment, inner wall 30 further comprises a filtering element that is adapted to selectively filter the wavelength of the energy transmitted from elements 24 and 32. By wall of illustration, and not limitation, transmitting element 24 may emit energy of wavelengths 400 nm to 750 nm and inner wall 30 may act as a filter such that only wavelengths of between 600 and 700 nm are allowed into cavity 20.

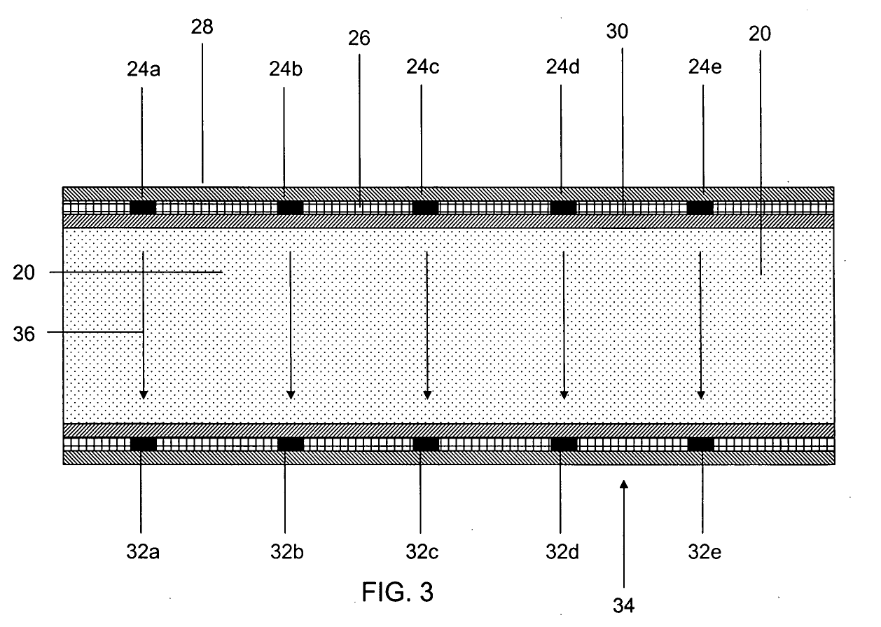

FIG. 3 is a cross sectional view of one embodiment of the invention wherein stent 34 comprises elements 24a to 24e which transmit electromagnetic energy 36 to receiving elements 32a to 32e. Stent 34 further comprises cavity 20, outer wall 28, inner wall 30 and middle layer 26. In the embodiment depicted in FIG. 3, the electromagnetic wave 36 is comprised of substantially parallel waves. In one embodiment, polarized light is used. In another embodiment, laser light is used. As is apparent from FIG. 3, the transmitting and receiving elements are aligned such that they are opposite to each other. Thus, in the embodiment depicted, transmitting element 24a will transmit energy 36 to receiving element 32a. The effect of the energy transmitted from transmitting element 24a will have a minimal impact on receiving elements 32b to 32e. In one embodiment, transmitting elements 24a to 24e are activated simultaneously. In another embodiment, transmitting elements 24a to 24e are activated sequentially. In yet another embodiment, transmitting elements 24a to 24e are activated sequentially in groups. For example, transmitting elements 24a and 24e transmit an energy wave, and afterwards, elements 24b and 24d transmit an energy wave.

In one embodiment of the invention, a baseline measurement is taken when cavity 20 is in its pristine state. When cavity 20 is filled with particles (not shown), these particles will absorb and/or scatter the energy 36 as energy 36 interacts with the particles. As such, the energy received by receiving element 32 will be less than the energy transmitted by transmitting element 24. When the environment within cavity 20 is relatively constant, a baseline measurement can be taken and the amount of energy that is successfully received by receiving element 32 can be recorded. As would be appreciated by those skilled in the art, the environment of a dynamic lumen undergoes minor changes. By way of illustration, and not limitation, as blood flows through a stent, the exact composition of the blood may not be precisely constant. As such, the amount of energy received by receiving element 32 may not be constant. Nevertheless, a sampling of data points over a period of time allows one to obtain a baseline measurement, as well as obtain a range of typical deviations from the baseline. Such deviations may be caused by the change in blood flow due to the beating of the heart, localized concentrations of red blood cells or other particles, and the like.

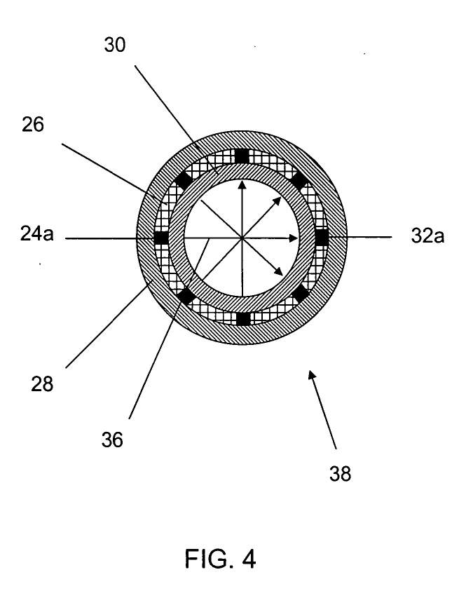

FIG. 4 depicts an end view of another embodiment similar to that depicted in FIG. 3. In the embodiment depicted, stent 38 comprises an inner wall 30, an outer wall 28, and a middle layer 26. Disposed within middle layer 26 are transmitting elements, such as 24a and receiving elements, such as 32a. In the embodiment shown in FIG. 4, transmitting element 24a transmits energy 36 which is sensed by receiving element 32a.

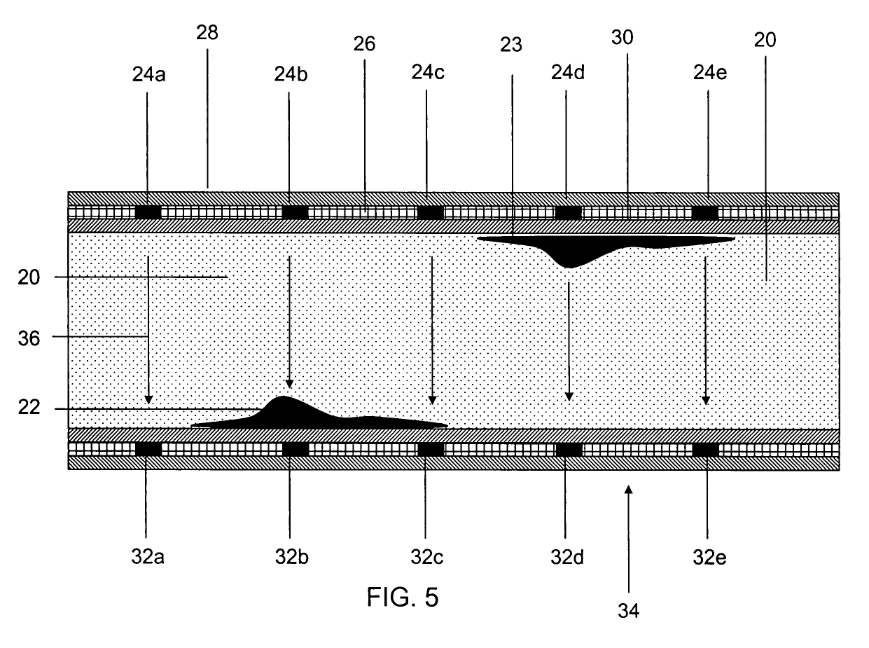

FIG. 5 is a cross section view of stent 34 depicting the restenosis of the stent. In the embodiment depicted, stent 34 comprises cavity 20, inner wall 30, outer wall 28, middle layer 26. Disposed within middle layer 26 are transmitting elements 24a to 24e and receiving elements 32a to 32e. As depicted in FIG. 5, stent 34 further comprises plaque 22 and 23. It is clear from the figure that the energy 36 that is transmitted from transmitting element 24a to receiving element 32a is not obstructed by plaque 22. As such, the intensity of energy 36 detected at 32a is equal to the intensity of the energy transmitted from 24a, minus the energy lost to the environment in cavity 20 (for example, scattering of energy due to the presence of blood in the cavity 20). The energy received by 32a is then compared to the baseline measurements taken when stent 38 was in pristine condition. In the embodiment depicted in FIG. 5, the energy received by 32a would be within the acceptable deviation limits as compared to the baseline measurements. In comparison, it is clear that the energy received by receiving element 32b is outside of the deviations expected, relative to the previously measured baseline. This is due to the additional scattering due to plaque 22. Similarly, element 32b would receive somewhat less energy, as compared to the baseline, due to the thin layer of plaque. The plaque need not be present at the receiving elements. For example, plaque 23 diminishes the energy received at receiving element 32d, even though it is at least partially covering transmitting element 24d.

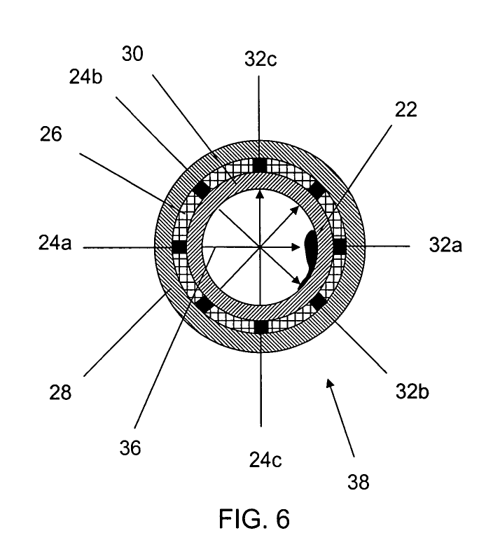

FIG. 6. depicts an end view of an embodiment similar to that shown in FIG. 5. Stent 38 comprises an inner wall 30, an outer wall 28, a middle layer 26, and plaque 22. Disposed within middle layer 26 are transmitting elements, such as 24a and receiving elements, such as 32a. In the embodiment shown in FIG. 6, transmitting element 24a transmits energy 36 which is sensed by receiving element 32a. It is clear from FIG. 6 that the energy received by receiving element 32a is less than the baseline due to the presence of plaque 22. Similarly, the energy received by receiving element 32b is less than the baseline, due to the thin layer of plaque 22. By contrast, the energy received at receiving element 32c would be within the typical deviation of the baseline value, as there is no significant scattering or absorbance of the energy due to a foreign body.

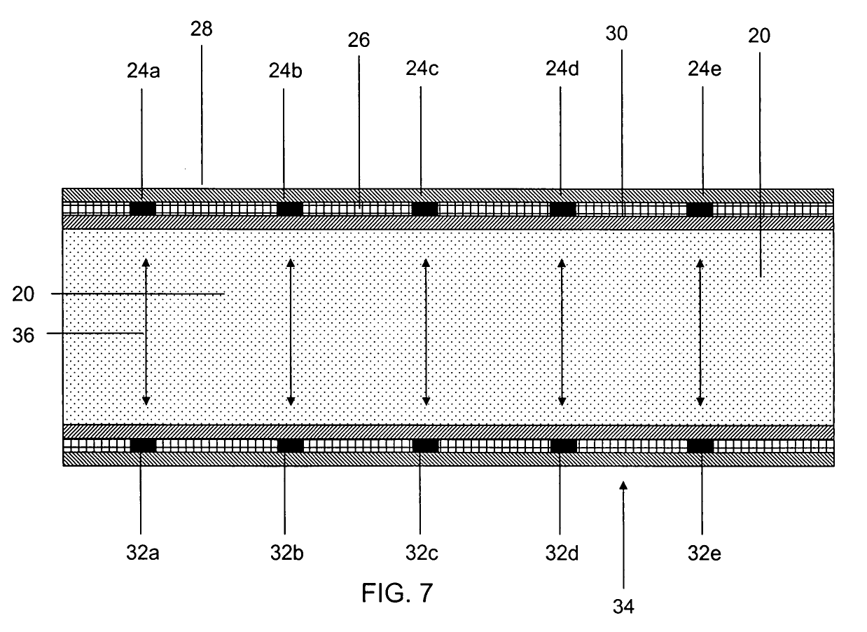

FIG. 7 is a cross sectional view of another embodiment of the invention which is similar that depicted in FIG. 3. In this embodiment, elements 24a to 24e and elements 32a to 32e function both as transmitting and receiving elements. Thus energy 36 may be transmitted in two directions.

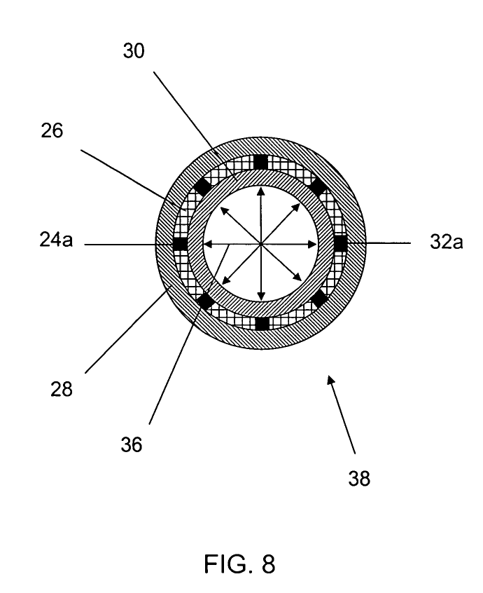

FIG. 8 is an end view of an embodiment similar to that depicted in FIG. 7. Elements 24a and 32a function as both transmitters and receivers of electromagnetic energy. In the embodiment depicted, the energy used comprises substantially parallel waves of energy. In another embodiment, the waves are non-parallel.

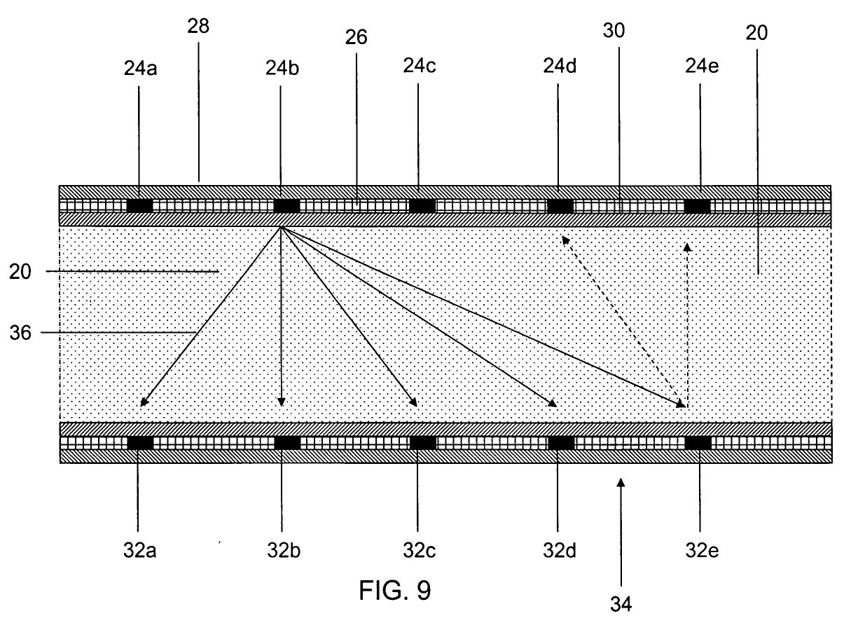

FIG. 9 is a cross sectional view of another embodiment of the invention which employs non-parallel waves of energy. In the embodiment depicted in FIG. 9, elements 24a to 24e and 32a to 32e are adapted to both transmit and receive energy. As shown in FIG. 9 element 24b broadcasts a wave of non-parallel wave energy, which is detected by receiving elements 32a to 32e. As would be apparent to one skilled in the art, the energy at receiving element 32b is most intense, but a certain portion of the energy is detected at the other receiving elements. In one embodiment, a portion of the energy is reflected off of the surface of the elements 32, and redirected back to elements 24. In one embodiment, none of the energy is redirected. In another embodiment, between 0.01% and 10% of the light is redirected. In another embodiment, between 10% and 50% of the light is redirected. In yet another embodiment, between 50% and 90% of the light is redirected. In the embodiment depicted, element 24b is functioning as a transmitter, while elements 24a, 24c to 24e, and 32a to 32e are all in “receive mode.” At another point in time, element 32d, for example, may be in “transmit mode” and the other elements in “receive mode.” In a similar manner, the elements can be sequentially activated and a map of the inner surface of stent 34 may be constructed. By conducting such measurements when the stent is in pristine condition, a baseline measurement may be obtained.

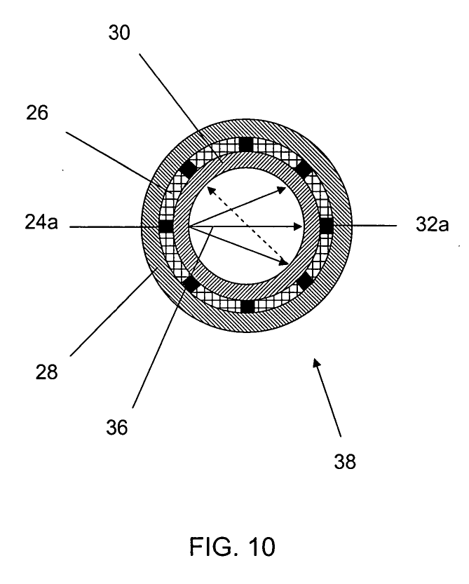

FIG. 10 is an end view of an embodiment of the device similar to that depicted in FIG. 9. Stent 38 comprises an inner wall 30, an outer wall 28, and a middle layer 26. Disposed within middle layer 26 are transmitting elements, such as 24a and receiving elements, such as 32a. In the embodiment shown in FIG. 10, transmitting element 24a transmits non-parallel energy 36 which is sensed most strongly by receiving element 32a, but is also sensed by the other receiving elements. A portion of the energy 36 is reflected off of inner wall 30 and detected by other elements. It is clear from the previous discussions that any obstructions, such as plaque depositions during restenosis, would be detected when a comparison is made to the baseline energy values.

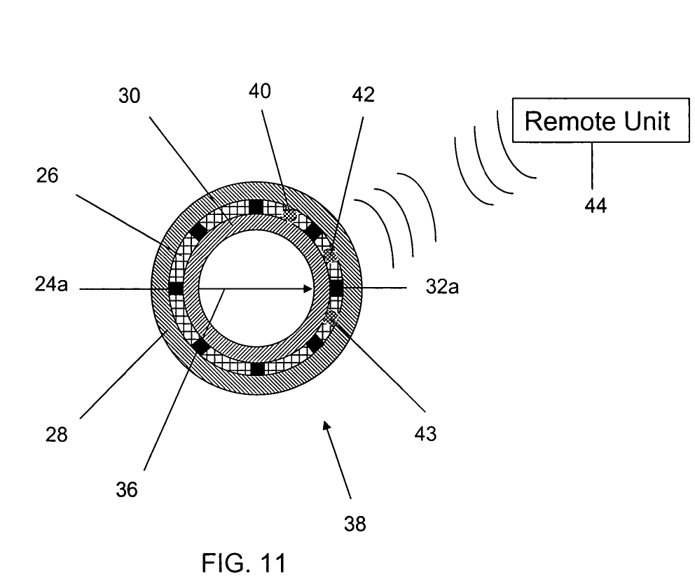

FIG. 11 is an end view of yet another embodiment of the invention, wherein power source 40 is shown. In the embodiment depicted, stent 38 comprises an inner wall 30, an outer wall 28, and a middle layer 26. Disposed within middle layer 26 are transmitting elements, such as 24a and receiving elements, such as 32a. In the embodiment shown in FIG. 11, transmitting element 24a transmits energy 36 which is sensed by receiving element 32a. In one embodiment, power source 40 is a convention power supply. Power source 40 provides a source of electrical power to elements 24 and 32. Thus, by way of illustration, one may use a lithium-iodine battery, and/or a battery that is chemically equivalent thereto. The battery used may, for example, have an anode of lithium or carbon and a cathode of iodine, carbon, or of silver vanadium oxide, and the like. By way of further illustration, one may use one or more of the batteries disclosed in U.S. Pat. No. 5,658,688, “Lithium-silver oxide battery and lithium-mercuric oxide battery,” U.S. Pat. No. 4,117,212, “Lithium-iodine battery,” and the like. In FIG. 11, power source 40 is disposed within middle layer 26. It is clear to those skilled in the art that the power source may be disposed elsewhere without deviating from the teaching of this invention.

FIG. 11. depicts an embodiment wherein remote unit 44 communicates with antenna 42. Antenna 42 is adapted to both transmit and receive signals from remote unit 44. In the embodiment shown, antenna 42 is disposed within middle layer 26. In another embodiment, the antenna is disposed in outer wall 28. In one embodiment, stent 38 comprises a microprocessor 43 that is operatively connected to transmitting element 24, receiving element 32, power source 40, and antenna 42. In one embodiment, the remote unit 44 is a data acquisition unit. In another embodiment, the remote unit 44 is a control unit. In yet another embodiment, the remote unit 44 is both a data acquisition unit and a control unit. For example, one may use the telemetry system disclosed in U.S. Pat. No. 5,843,139, “Remotely operable stent.” By way of further illustration, one may use the remote system disclosed in U.S. Pat. No. 5,843,139 and the like. Acoustic energy may also be employed. See, for example, U.S. Pat. No. 6,170,488, “Acoustic-based remotely interrogated diagnostic implant device and system.”

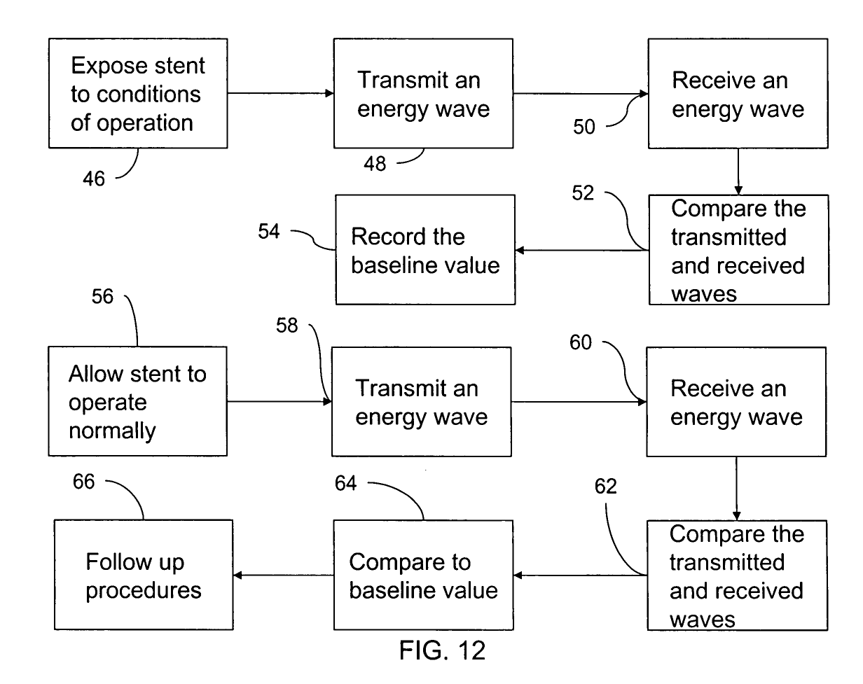

FIG. 12 is a flowchart that illustrates one process of the invention. In steps 46 to 54, a baseline measurement is obtained. In step 46, the stent is exposed to the conditions of operation. By way of illustration, if the stent is to be disposed in a blood vessel, then blood is allowed to flow through the stent. In step 48, a wave is transmitted across the lumen of the stent. The intensity of the wave is recorded in the microprocessor of the stent. In step 50, the energy wave is received. Step 52 then compares the intensity of the wave received in step 50 to the intensity of the wave transmitted in step 48. In step 54, this comparison value (i.e. the baseline value) is recorded in the stent's microchip. Alternatively or additionally, the recorded value may be transmitted to a remote unit (see, for example, FIG. 11). In one embodiment, several baseline values are recorded, and an acceptable “baseline range” is obtained.

In steps 56 to 66 illustrated in FIG. 12, the stent performs a diagnostic procedure to detect any possible restenosis that may have occurred since the baseline measurement was recorded. In step 56, the stent is allowed to operate normally for a period of time. In step 58, a wave is transmitted across the lumen of the stent. The intensity of the wave is recorded in the microprocessor of the stent. In step 60, the energy wave is received. Step 62 then compares the intensity of the wave received in step 60 to the intensity of the wave transmitted in step 58. Step 64 compares the value obtained from step 62 to the baseline (or baseline range). Step 66, which is optional, is a step the stent performs depending on the value obtained in step 64.

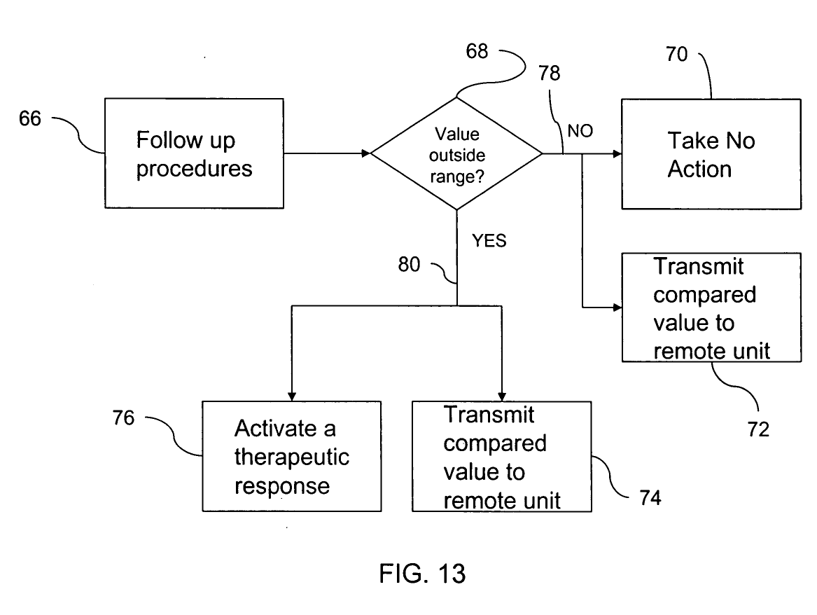

FIG. 13 is a flow chart that depicts step 64 in more detail. In step 68, the value obtained from step 64 is compared to the baseline obtained in step 54. If the value is within an acceptable range, then path 78 will be followed. In one embodiment, step 70 is executed, wherein no action is taken. In another embodiment, step 72 is followed, wherein the value obtained in step 64 is transmitted to a remote unit. If the value obtained in step 64 is outside of an acceptable range, then path 80 is followed. In one embodiment, not shown, no action is taken. In another embodiment, step 74 is taken, wherein the value obtained in step 64 is transmitted to a remote unit (step 74). In another embodiment, a therapeutic response is triggered (step 76). In yet another embodiment, both step 74 and 76 are executed.

A number of therapeutic responses may be triggered. In one embodiment, an anticoagulant is released to counteract restenosis. In another embodiment, a therapeutic agent is released. In another embodiment, the therapeutic agent released acts to counteract restenosis. Reference may be had, for example, to U.S. Pat. Nos. 5,865,814; 6,613,084; 6,613,082; 6,656,162; 6,589,546; 6,545,097; 6,491,666; 6,379,382; 6,344,028; 5,865,814 and the like. The content of each of these patents is hereby incorporated by reference. As would be apparent to one skilled in the art, the release of the agent may be triggered remotely by remote unit 44, and need not necessarily be coupled to the value obtained in step 64.

In another embodiment, the therapeutic response comprises a release of energy of sufficient intensity to counteract restenosis. Reference may be had to U.S. Pat. Nos. 6,709,693; 6,200,307; 5,964,751 and the like. The content of each of these patents is hereby incorporated by reference.

The telemetry means taught above may also be used to reprogram microprocessor 43 in vivo. Thus, it is possible to trigger the remote activation of steps 46 to 54 without removing the stent from the body. Additionally or alternative, a range of acceptable deviation values may be remotely programmed or reprogrammed via remote unit 44.

As would be apparent to one skilled in the art, a variety of forms of energy may be used with the instant invention. In one embodiment, vibrational energy is used. In another embodiment, acoustic energy is used. In one embodiment, a piezoelectric crystal is used to generate the acoustic energy. In another embodiment electromagnetic radiation is used. In one embodiment, the electromagnetic energy used is vacuum UV radiation. In another embodiment, the energy used is near UV energy. In another embodiment the energy used is visible light. In another embodiment the energy used is infrared radiation. In yet another embodiment, the energy used is radio frequency energy. In one embodiment, the energy used has a wavelength between about 400 nm and about 750 nm. In another embodiment, the energy used has a wavelength between about 600 nm and about 700 nm. In another embodiment, the wavelength of the energy is between about 1 nm and about 400 nm. In another embodiment, the wavelength of energy used is between about 750 nm and about 3 μm. In yet another embodiment, the wavelength of energy used is between about 3 μm and 30 μm. In yet still another embodiment, the wavelength of energy used is between 30 μm and 1 mm. In another embodiment, the wavelength of energy used is between about 1 m and about 10⁵ m, and preferably between 1 m and 10³ m. In yet another embodiment, the wavelength of energy used is between 10⁻³ m and 1 m.

Numerous methods for the manufacturing and implantation of stents and modified stents are well known to those skilled in the art. Reference may be had to U.S. Pat. Nos. 6,527,919; 6,190,393; 6,124,523; 6,096,175 and the like.

It is, therefore, apparent that there has been provided, in accordance with the present invention, a method and apparatus for the detection of restenosis within a stent. While this invention has been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of the appended claims.

I claim:

1. A medical apparatus comprising a stent, a transmitter operatively configured to generate an electromagnetic wave to produce a transmitted wave, a receiver operatively configured to receive said transmitted wave to produce a received wave, and a processor operatively configured to compare said transmitted wave and said received wave to produce a compared value.

2. The medical apparatus as recited in claim 1 wherein said processor is operatively configured to compare said compared value to a baseline value to produce a corrected value.

3. The medical apparatus as recited in claim 2, wherein said corrected value is indicative of restenosis within said stent.

4. The medical apparatus as recited in claim 3, wherein a therapeutic response is triggered when said corrected value deviates from said baseline value.

5. The medical apparatus as recited in claim 3 wherein said processor is operatively configured to transmit said corrected value to a remote unit, wherein said remote unit is external to said stent.

6. The medical apparatus as recited in claim 5, wherein said processor is operatively configured to receive a transmission from said remote unit.

7. The medical apparatus as recited in claim 3, wherein said transmitted energy is comprised of substantially parallel energy.

8. The medical apparatus as recited in claim 7, wherein said transmitter is a vertical cavity surface emitting laser.

9. The medical apparatus as recited in claim 3, wherein said transmitted energy is comprised of substantially non-parallel energy.

10. The medical apparatus as recited in claim 3 wherein said stent is disposed within a living organism.

11. The medical apparatus as recited in claim 3 wherein said transmitter and said receiver are at least partially disposed within said stent.

12. The medical apparatus as recited in claim 3 wherein said transmitted wave is an electromagnetic wave with a wavelength from about 400 nm to about 750 nm.

13. The medical apparatus as recited in claim 3 wherein said transmitted wave has a wavelength from about 600 nm to about 700 nm.

14. A process for detecting restenosis of a medical apparatus comprising the steps of
a. obtaining a baseline measurement for a stent while said stent is exposed to a first environmental condition,
b. expositing said stent to a second environmental condition,
c. transmitting energy from a transmitting element disposed within said stent to produce transmitted energy,
d. receiving said transmitted energy at receiving element disposed within said stent to produce received energy, and
e. determining the degree of stenosis of said stent in said second environmental condition based on the comparison of the values of said transmitted energy, said received energy, and said baseline measurement.

15. The process as recited in claim 14, wherein said stent is disposed within a living organism.

16. A medical apparatus comprising a stent, means for generating an energy wave to produce a transmitted wave, means for receiving said transmitted wave to produce a received wave, and means for comparing said transmitted wave and said received wave to produce a compared value.

17. The medical apparatus as recited in claim 16 wherein said means for comparing said transmitted wave and said received wave further comprises means for comparing said compared value to a baseline value to generate a corrected value.

18. The medical apparatus as recited in claim 17, wherein said corrected value is indicative of restenosis within said stent.

19. The medical apparatus as recited in claim 16 wherein said transmitted wave is a vibrational wave.

20. The medical apparatus as recited in claim 18, wherein said transmitted wave is an electromagnetic wave.
